(19) United States
(12) Patent Application Publication
(10) Pub. No.: US 2016/0206792 A1
(43) Pub. Date: Jul. 21, 2016

(54) SELF ADHERING SUCTION PORT PAD SECUREMENT DEVICE FOR USE WITH A NEGATIVE PRESSURE WOUND HEALING DRAPE OR DRESSING

(71) Applicant: George Medical, LLC, Wheaton, IL (US)

(72) Inventor: George Worthley, Wheaton, IL (US)

(73) Assignee: George Medical, LLC, Wheaton, IL (US)

(21) Appl. No.: 14/977,182

(22) Filed: Dec. 21, 2015

Related U.S. Application Data
(60) Provisional application No. 62/125,210, filed on Jan. 16, 2015.

(51) Int. Cl. A61M 1/00; A61F 13/02

(52) U.S. Cl. CPC A61M 1/0088; A61F 13/0216; A61F 13/0263; A61F 2013/00289

(57) ABSTRACT

A securement device may have a release strip, a release layer, a film having an adhesive and/or a back layer. The release strip and/or the release layer may be removable from the adhesive. The release strip may have a first aperture that may extend through the release layer. The film and/or the adhesive may have a second aperture that may extend through the film and/or the adhesive. The release layer may be positioned between the release strip and the film. The release layer may have a third aperture that may be larger in diameter than the first aperture and/or the second aperture. The release strip may be removed from the securement device which may expose the adhesive. A suction port may be adhered to the adhesive. The securement device and/or the suction port may be secured to a dressing which may be used in negative pressure wound therapy.

### CROSS-REFERENCE TO RELATED APPLICATIONS

This application claims the benefit of U.S. Provisional Application No. 62/125,210, filed Jan. 16, 2015 (01/16/2015), the disclosure of which is hereby incorporated in its entirety.

### BACKGROUND OF THE INVENTION

The present invention generally relates to a securement device that may have a release strip that may be removable from the securement device and/or a method for assembling the same. More specifically, the present invention relates to a securement device that may have a release strip that may be removable from the securement device and/or a method for assembling the same that may be secured to a dressing for negative pressure wound therapy (NPWT).

It is generally known to use dressings for treatment and/or coverings of wounds on a patient. Known dressings are generally constructed from a film, foam, fabric and/or combination thereof and/or may have an adhesive which may secure the dressing to skin of the patient that may surround the wound. Securement devices may interface with the dressing and/or may secure a suction port to the dressing which may create a seal over the wound and/or may ensure a vacuum.

It is also generally known that the dressings and/or the securement devices may be used in NPWT which may enhance growth of tissue of the wound and/or may enhance healing of the wound. When administering NPWT on the patient, the dressing may cover the wound and/or the dressing may have an adhesive to secure the dressing to the skin that may be adjacent to the wound and/or that may surround the wound. Further, the securement device may have an adhesive to secure the securement device to the dressing and/or the suction port and/or to create the seal. Negative pressure may be applied to the wound, such as, for example, by using a vacuum, which may remove fluids, exudates, infectious material and/or the like from the wound. The fluids, the exudates and/or the infectious material may be drained through tubing that may be connected to a suction port which may be secured to the dressing and/or to the securement device.

Individuals and/or machines may have difficulty removing known layers of the securement device from the securement device to expose an adhesive that may secure the suction port to the securement device. Such difficulty may increase a likelihood of damaging the securement device. If the securement device is damaged, such as, for example, having a rip and/or a hole, the seal may not be created and/or the vacuum may not remove the fluids, exudates, infectious material and/or the like from the wound. Further, the known securement devices may be costly to manufacture.

A need, therefore, exists for a securement device that may have a release strip and/or a method for assembling the same where the release strip may be removable from the securement device in a manner that may reduce a likelihood of damaging the securement device. Further, a need exists for a securement device that may have a release strip and/or a method for assembling the same where the release strip may reduce manufacturing costs of the securement device. Moreover, a need exists for a securement device that may have a release strip and/or a method for assembling the same where the release strip may be removed quickly and/or easily from the securement device.

### SUMMARY

The present invention generally relates to a securement device and/or a method for assembling the same. More specifically, the present invention relates to a securement device and/or a method for assembling the same that may have a release strip that may be removable from the securement device.

To this end, in an embodiment of the present invention, a securement device is provided. The securement device may have a release strip, a film, and/or a release layer. The release strip may have a first aperture that extends through the release strip and may have a perimeter defined by a first edge, a second edge, a third edge and a fourth edge wherein the first edge is positioned opposite to the second edge and the third edge is positioned opposite to the fourth edge and further wherein the first edge and the second edge are generally perpendicular to the third edge and the fourth edge. The film may have a top surface with an adhesive and bottom surface positioned opposite to the top surface wherein the film has a second aperture that extends through the film wherein the first aperture and the second aperture are substantially the same size. The release layer may be secured to the adhesive wherein the release layer is between the release strip and the film and has a third aperture that extends through the release layer wherein the third aperture is larger than the first aperture and the second aperture wherein the release layer covers the film and is aligned on the film so that the adhesive has an exposed region that extends from the second aperture to the third aperture and further wherein the release strip is secured to the exposed region of the adhesive.

In an embodiment, the securement device may have tabs secured to the adhesive on the film wherein the tabs are between the release layer and the film.

In an embodiment, the film may be constructed of a material that is transparent.

In an embodiment, the securement device may have a back layer having a fourth aperture that extends through the back layer wherein the back layer is secured to the bottom surface of the film and further wherein the film is positioned between the back layer and the release layer.

In an embodiment, the securement device may have a suction port having a dome that is generally hemispherical in shape and a flange that extends from the dome wherein the flange is secured to the exposed region of the adhesive.

In an embodiment, the securement device may have tubing that is adjacent to the film wherein the tubing extends from the second aperture.

In another embodiment of the present invention, a method for assembling a securement device for use in negative pressure wound therapy wherein the securement device may have a release strip, a release layer, a film with an adhesive and a back layer, wherein the release strip, the release layer, the film, the adhesive and the back layer are generally planar and generally parallel with respect to each other is provided. The method may have the step of coating a top surface of the film with an adhesive wherein the film has a first aperture that extends through the film. The method may have the step of attaching the back layer to the film wherein the back layer covers the film. The method may have the step of securing the release layer to the adhesive wherein the release layer has a second aperture that extends through the release and that is larger than the first aperture and further wherein the release layer covers the adhesive such that a ring of the adhesive is exposed between the first aperture and the second aperture. The method may have the step of securing the release strip to the ring of the adhesive wherein the release strip has a section that is unsecured to the ring of adhesive and that contacts the release layer.

In an embodiment, the method may have the step of grasping the section of the release strip.

In an embodiment, the method may have the step pulling the section of the release strip in a direction away from the film to remove the release strip.

In an embodiment, the method may have the step attaching a suction port to the ring wherein the suction port has a flange that covers the ring.

In an embodiment, the method may have the step removing the release layer from the film.

In an embodiment, the method may have the step peeling the back layer from the film.

In another embodiment of the present invention, a securement device for use in negative pressure wound therapy is provided. The securement device may have a film, a release layer, a release strip and/or a back layer. The film may have a top surface with an adhesive and bottom surface positioned opposite to the top surface wherein the film has a first perimeter and has a first aperture having a first diameter wherein the first aperture extends through the film. The release layer may be secured to the film wherein the release layer has a second perimeter and is substantially parallel to the film wherein the release layer has a second aperture that extends through the release layer wherein the first perimeter and the second perimeter are aligned with respect to each other wherein the second aperture has a second diameter that is greater than the first diameter so that the film has a ring of adhesive that is exposed between the first aperture and the second aperture. The release strip may have a third aperture that extends through the release strip and may have a third perimeter defined by a first edge, a second edge, a third edge and a fourth edge wherein the first edge is positioned opposite to the second edge and the third edge is positioned opposite to the fourth edge and further wherein the first edge and the second edge are generally perpendicular to the third edge and the fourth edge wherein the first edge and the second edge extend along the second perimeter wherein the release strip contacts the release layer and is adhered to the ring of adhesive and further wherein the release strip has a section that extends beyond the ring of adhesive and is unsecured to the ring of adhesive. The back layer may have a fourth aperture that extends through the back layer wherein the back layer is secured to the film wherein the film is between the back layer and the release layer and further wherein the back layer has a fourth perimeter that is aligned with respect to the second perimeter.

In an embodiment, the securement device may have tabs secured to the adhesive on the top surface of the film wherein the tabs are positioned on opposite ends of the film and further wherein the tabs are between the release layer and the back layer.

In an embodiment, the securement device may have a suction port having a dome and a flange that extends from the dome wherein the flange is substantially parallel to the film and is adhered to the ring.

In an embodiment, the the film may be constructed from polyurethane.

In an embodiment, the ring may be generally circular. In an embodiment, the back layer may be kiss cut so that the back layer is divided between a first side and a second side. In an embodiment, the securment device may have tubing that may be generally cylindrical and that may be adjacent to the film.

In an embodiment, the first perimeter and the second perimeter may be substantially shaped as an oval.

It is, therefore, an advantage of the present invention to provide a securement device that may have a release strip that may be removable from the securement device and/or a method for assembling the same.

Another advantage of the present invention is to provide a securement device that may have a release strip that may be removable from the securement device and/or a method for assembling the same that may reduce a likelihood of damage to a film of the securement device.

Further, an advantage of the present invention is to provide a securement device that may have a release strip that may be removable from the securement device and/or a method for assembling the same that may reduce manufacturing costs of the securement device.

Moreover, an advantage of the present invention is to provide a securement device that may have a release strip that may be removable from the securement device and/or a method for assembling the same that may allow a suction port to be secured quickly and/or easily to the securement device.

Yet another advantage of the present invention is to provide a securement device that may have a release strip that may be removable from the securement device and/or a method for assembling the same that may be secured to a dressing that may be used in negative pressure wound therapy.

Moreover, an advantage of the present invention is to provide a securement device that may have a release strip that may be removable from the securement device and/or a method for assembling the same that may allow the release strip to be quickly and/or easily removed from the securement device.

Another advantage of the present invention is to provide a securement device that may have a release strip that may be removable from the securement device and/or a method for assembling the same wherein a release layer of the securement device may display patient identification information, manufacturer information and/or the like.

Additional features and advantages of the present invention are described in, and will be apparent from, the detailed description of the presently preferred embodiments and from the drawings.

### BRIEF DESCRIPTION OF THE DRAWINGS

FIG. 1 illustrates a perspective view of a securement device in an embodiment of the present invention.

FIG. 2 illustrates a perspective view of a securement device in an alternate embodiment of the present invention.

FIG. 3 illustrates an exploded view of a securement device in an embodiment of the present invention.

FIG. 4 illustrates a perspective view of a securement device with a release strip being removed from the securement device in an embodiment of the present invention.

FIG. 5 illustrates a perspective view of a securement device in an embodiment of the present invention with a suction port.

FIG. 6. illustrates a perspective view of a securement device in an embodiment of the present invention with a suction port secured to the securement device.

### DETAILED DESCRIPTION

The present invention generally relates to a securement device and/or a method for assembling the same. The securement device may have a release strip that may be removable from the securement device and/or that may be removed to expose an adhesive. A suction port may be secured to the adhesive. A release layer on the securement device may be removable from the securement device and/or may be removed to expose the adhesive. The securement device may be adhered to a dressing (not shown) that may be used in negative pressure wound therapy (NPWT) administered to an individual having a wound (not shown).

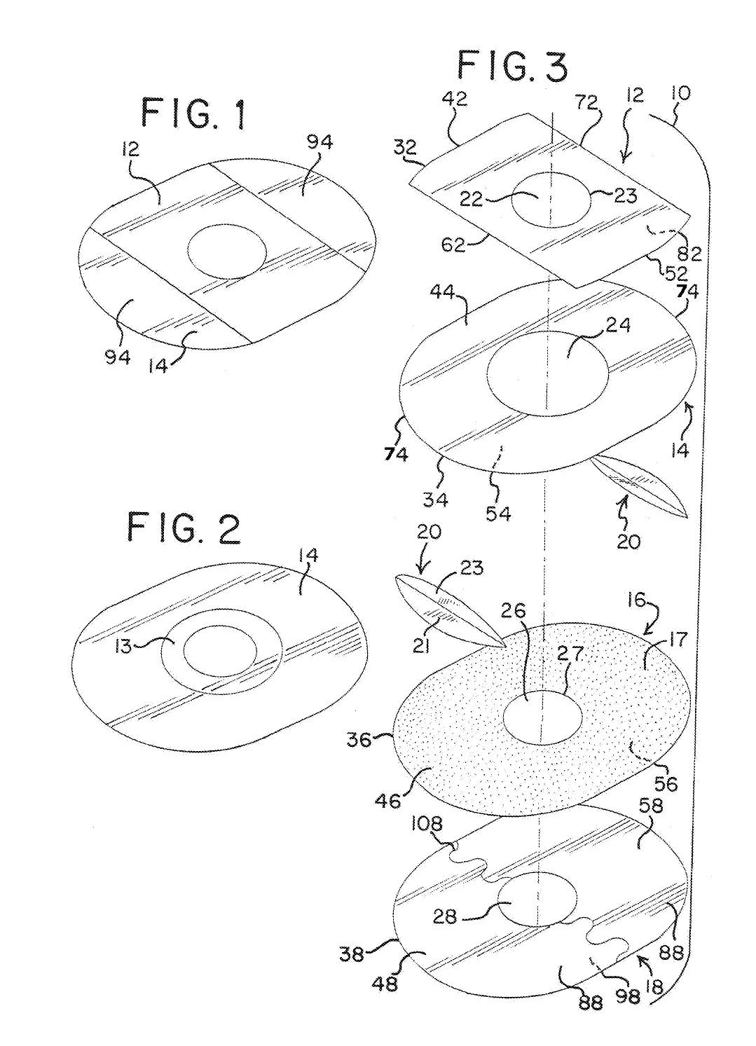

Referring now to the drawings wherein like numerals refer to like parts, FIGS. 1-6 show a securement device 10 in an embodiment of the present invention. As shown in FIGS. 1-6, the securement device 10 may be generally shaped as an oval. However, it should be understood that the securement device 10 may be formed in any shape as known to one having ordinary skill in the art. The securement device may have a release strip 12, a release layer 14, a film 16, an adhesive 17, a back layer 18 and/or tabs 20.

As shown in FIGS. 1, 3 and 4, a first bottom surface 82 of the release strip 12 may be secured to the film 16 by the adhesive 17 and/or may be connected to the release layer 14. The release strip 12 may be constructed from paper and/or any material known to one having ordinary skill in the art that may be secured to the adhesive 17 and/or may reduce damage to the film 16. In an embodiment, the release strip 12 may be constructed from a clay coated bleached kraft paper that may have a basis weight between fifty pounds and sixty pounds. In an embodiment, a silicone release coating may be located on the release strip 12 and/or on the first bottom surface 82. The release strip 12 may be generally shaped as a rectangle and/or may have a first perimeter 32 defined by a first edge 42, a second edge 52, a third edge 62 and/or a fourth edge 72. The first edge 42 may be positioned opposite to the second edge 52 and/or may be generally parallel with respect to the second edge 52. The third edge 62 may be positioned opposite to the fourth edge 72 and/or may be generally parallel with respect to the fourth edge 72. The first edge 42 and/or the second edge 52 may be generally perpendicular with respect to the third edge 62 and/or the fourth edge 72. Manufacturing costs of the securement device 10 may be reduced as a result of the shape of the release strip 12. A first aperture 22 may be cut into the release strip 12 and/or may extend through the release strip 12. In an embodiment, the first aperture 22 may be generally circular.

The film 16 may be constructed from polyurethane and/or the like and/or may be transparent and/or translucent. In an embodiment, the film 16 may be constructed from a monolithic translucent matte finish polyurethane film which may have a high vapor transmission rate. In an embodiment, the film 16 may have a thickness of approximately 0.001 inches. The adhesive 17 may be coated on a first top surface 46 of the film 16, for example, by a transfer coating process. In an embodiment, the adhesive 17 may be constructed from an acrylic which may be cured by ultraviolet light. In an embodiment, the adhesive 17 may have a thickness of approximately 0.001 inches and/or may be constructed from a material which may safely contact and/or adhere to human skin. As shown in FIG. 3, a second aperture 26 may be cut into the film 16 and/or the adhesive 17 and/or the second aperture 26 may extend through the film 16 and/or the adhesive 17. In an embodiment, the second aperture 26 may be generally circular. When the securement device 10 is assembled, the second aperture 26 may be aligned with the first aperture 22 such that the first aperture 22 and/or the second aperture 26 may have a common axis of symmetry and/or may be centered with respect to each other. In an embodiment, the film 16 may have a second perimeter 36 that may be generally shaped as an oval. In an embodiment, the film 16 may have a first surface area that may be greater than a second surface area of the release layer 14.

In an embodiment, the film 16 may be extruded onto the back layer 18. The film 16 may be secured to the back layer 18, for example, by a heat bonding process. In an embodiment, the back layer 18 may be constructed from a bleached kraft paper. In an embodiment, a second top surface 88 of the back layer 18 may have a silicone coated polyethylene layer. In an embodiment, a second bottom surface 98 may have a silicone coated layer. A third aperture 28 may be cut into the back layer 18 and/or may extend through the back layer 18. In an embodiment, the third aperture 28 may be generally circular. When the securement device 10 is assembled, the third aperture 28 may be aligned with the first aperture 22 and/or the second aperture 26 such that the first aperture 22, the second aperture 26 and/or the third aperture 28 may have a common axis of symmetry and/or may be centered with respect to each other.

As shown in FIG. 3, the back layer 18 may be kiss cut and, as a result, the back layer 18 may be separated into a first side 48 and/or a second side 58. The back layer 18 may have a dividing line 108 that may indicate a separation of the first side 48 from the second side 58. In an embodiment, the back layer 18 may have a third perimeter 38 that may be generally shaped as an oval. When the securement device 10 is assembled, the release strip 12, the release layer 14, the film 16, and/or the back layer 18 may be substantially parallel with respect to each other.

In an embodiment, the release layer 14 may be constructed from paper and/or the like and/or may have a fourth perimeter 34 that may be generally shaped as an oval. Patient identification information, manufacturer information and/or the like may be located on a third top surface 44 of the release layer 14. When the secumrent device 10 is assembled, the release layer 14 and/or the back layer 18 may protect the film 16 from damage, such as, punctures and/or rips. In an embodiment, the release layer 14 may be constructed from a clay coated bleached kraft paper that may have a basis weight between approximately fifty pounds and sixty pounds. In an embodiment, a silicone release coating may be located on the release layer 14 and/or a third bottom surface 54. As shown in FIG. 3, the release layer 14 may have a fourth aperture 24 that may be cut into the release layer 14 and/or that may extend through the release layer 14. The fourth aperture 24 may be generally circular and/or may have a diameter that may be greater than a diameter of the first aperture 22 and/or may be greater than a diameter of the second aperture 26. The fourth aperture 24 may be sized so that a ring 64 may be formed when the release layer 14 is positioned on the film 16 and/or the adhesive 17. The ring 64 may be a portion of the adhesive 17 and/or the film that may not be covered by the release layer 14 when the release layer 14 is positioned on the film 16. The ring 64 may be exposed when the release layer 14 is positioned on the film 16 and/or may be exposed when the release strip 12 is removed from the release layer 14.

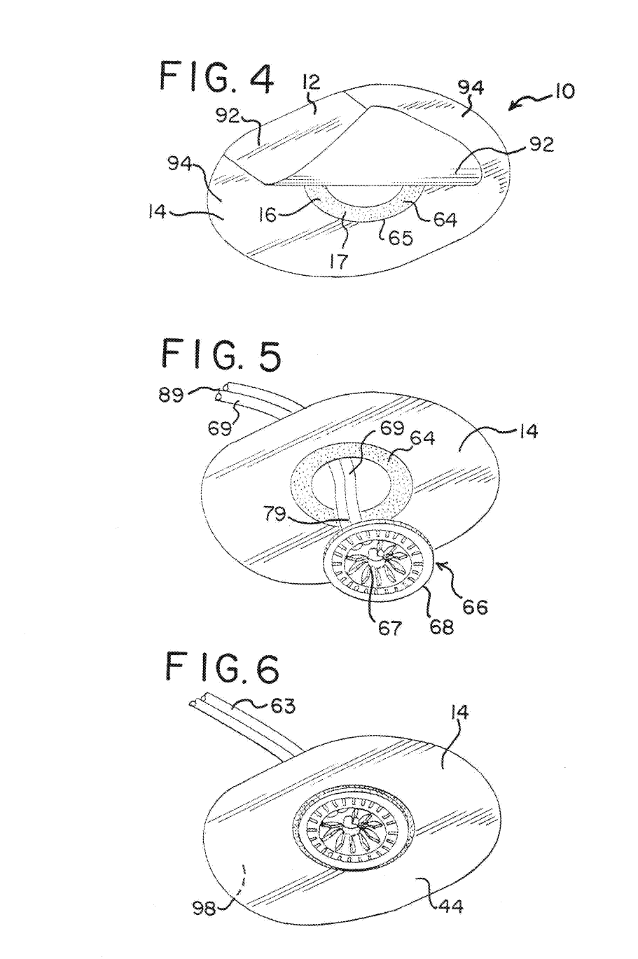

As shown in FIGS. 1 and 4, a section 94 of the release layer 14 may be uncovered by the release strip 12 when the release strip 12 is positioned on the release layer 14. The section 94 may extend from the third edge 62 to the fourth perimeter 34 and/or may extend from the fourth edge 72 to the fourth perimeter 34.

In an embodiment, the release strip 12 may be sized and/or shaped so that a section 92 of the release strip 12 may extend beyond a first exterior 65 of the ring 64. The section 92 may not be adhered to the adhesive 17 and/or the film 16. The section 92 may extend from the first exterior 65 of the ring 64 to the first edge 42, the second edge 52, the third edge 62 and/or the fourth edge 72 of the release strip 12. The section 92 may be unsecured to the adhesive 17 which may allow for a less difficult removal of the release strip 12 from the adhesive 17 and/or from the film 16 than removal of the release strip 12 without the section 92. As shown in FIG. 4, the release strip 12 may be removed from the securement device 10 by grasping the section 92 and/or lifting the section 92 in a direction away from the release layer 14 and/or the film 16. Removing the release strip 12 from the release layer 14 may expose the ring 64. As shown in FIG. 2, in an alternate embodiment, a release strip 13 may cover the ring 64 and/or may be substantially the same size as the ring 64.

To assemble the securement device 10, the film 16 may be extruded onto the back layer 18 such that a fourth bottom surface 56 of the film 16 may be secured to the second top surface 88 of the back layer 18. The second bottom surface 98 of the back layer 18 may be positioned opposite to the second top surface 88 and/or may face in a direction away from the film 16. The tabs 20 may be secured to ends 74 of the film 16 and/or the adhesive 17. The ends 74 may be located on opposite sides of the fourth perimeter 34 of the film 16. The tabs 20 may have a first portion 21 that may be secured to the adhesive 16 and/or may have a second portion 23 that may extend in an upward direction from the film 16.

The third bottom surface 54 of the release layer 14 may be secured to the adhesive 17 and/or the film 16. The release layer 14 may be unsecured to the adhesive 17 and/or the film 16 at the ends 74. After securing the release layer 14 to the adhesive 17 and/or to the film 16, the ring 64 of the adhesive 17 may be exposed. The release strip 12 may be secured to the film 16 and/or may contact the release layer 14 by attaching the release strip 12 to the ring 64. When the release strip 12 is secured to the film 16, a second exterior 25 of the first aperture 22 and/or a third exterior 27 of the second aperture 26 may be aligned with respect to each other. After securing the release strip 12 to the ring 64, the section 92 may contact the release layer 14. When the securement 10 is assembled, the second perimeter 36, the third perimeter 38 and/or the fourth perimeter 34 may be substantially aligned with respect to each other.

After the release strip 12 is removed from the securement device 10 which may expose the ring 64, a suction port 66 may be secured to the securement device 10. The suction port 66 may have a dome 67 and/or a flange 68. The dome 67 may be generally hemispherical in shape and/or the flange 68 may extend from the dome, for example, radially. In an embodiment, tubing 69 may be connected to and/or integrally formed with the suction port 66. The tubing 69 may have a first end 79 and/or a second end 89 that may be positioned opposite to the first end 89. As shown in FIG. 5, the first end 79 may be connected to and/or integrally formed with the dome 67 and/or the second end 89 may be connected to a vacuum (not shown) and/or to a canister (not shown). The vacuum may apply the negative pressure to the wound and/or the canister may store fluids, exudates, infectious material and/or the like that may be drained from the wound during NPWT. As shown in FIGS. 5 and 6, the tubing 69 may be bifurcated. However, in an embodiment, the tubing 69 may be constructed from a single tube (not shown). To secure the suction port 66 to the securement device 10, the tubing 69 may be inserted through the second aperture 26, the third aperture 28 and/or the fourth aperture 24, as shown in FIG. 5. The flange 68 may be aligned with the ring 64 and/or pressure may be applied to the flange 68 to adhere the flange 68 to the film 16 and/or the adhesive 17.

After securing the suction port 66 to the film 16 and/or the adhesive 17, the release layer 14 may be removed from the film 16 which may expose the adhesive 17. Another individual, such as a caregiver administering NPWT to the individual, may transport the securement device while the adhesive 17 is exposed. The other individual may grasp the securement device 10 by the tabs 20 so as not to touch the adhesive 17 and/or not to damage the film 16 when holding and/or transporting the securement device 10. The securement device 10 may then be interfaced with the dressing that may be secured to and/or may cover the wound of the individual. To interface the dressing with the securement device 10, the suction port 60 may be aligned with a hole (not shown) that may be cut into the dressing. Aligning the suction port 66 with the hole may provide a pathway for the fluids, the exudates, the infectious material and/or the like to be removed from the wound during NPWT and/or to be transferred to the canister. When the securement device 10 is interfaced with the dressing, the adhesive 17 may contact the dressing to secure the securement device 10 to the dressing.

The tabs 20 may be removed by pulling the portion 23 of the tabs 20 in a direction away from the film 16. A seal may then be created by applying pressure to an area adjacent to and/or surrounding the wound of the individual. The tubing 69 may be connected to the vacuum and/or the canister, and negative pressure may be applied to the wound. The fluids, the exudates, the infectious material and/or the like may be drained into the canister using the vacuum. The back layer 18 may be removed by peeling the first side 48 and/or the second side 58 in a direction away from the wound. As a result of the back layer 18 being kiss cut, the back layer 18 may be removed from the securement device 10 without interfering with the connection of the tubing 69. For example, the first side 48 and/or the second side 58 may be removed from the securement device 10 without disconnecting the tubing 69 from the suction port 66 and/or from the vacuum.

The other individual may then administer NPWT to the individual. The fluids, the exudates, the infectious material and/or the like may be removed from the wound and/or may be transferred through the tubing to the canister using the vacuum.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

I claim:

1. A securement device comprising:
a release strip having a first aperture that extends through the release strip and having a perimeter defined by a first edge, a second edge, a third edge and a fourth edge wherein the first edge is positioned opposite to the second edge and the third edge is positioned opposite to the fourth edge and further wherein the first edge and the second edge are generally perpendicular to the third edge and the fourth edge;
a film having a top surface with an adhesive and bottom surface positioned opposite to the top surface wherein the film has a second aperture that extends through the film wherein the first aperture and the second aperture are substantially the same size; and
a release layer secured to the adhesive wherein the release layer is between the release strip and the film and has a third aperture that extends through the release layer wherein the third aperture is larger than the first aperture and the second aperture wherein the release layer covers the film and is aligned on the film so that the adhesive has an exposed region that extends from the second aperture to the third aperture and further wherein the release strip is secured to the exposed region of the adhesive.

2. The securement device of claim 1 further comprising:
tabs secured to the adhesive on the film wherein the tabs are between the release layer and the film.

3. The securement device of claim 1 wherein the film is constructed of a material that is transparent.

4. The securement device of claim 1 further comprising:
a back layer having a fourth aperture that extends through the back layer wherein the back layer is secured to the bottom surface of the film and further wherein the film is positioned between the back layer and the release layer.

5. The securement device of claim 1 further comprising:
a suction port having a dome that is generally hemispherical in shape and a flange that extends from the dome wherein the flange is secured to the exposed region of the adhesive.

6. The securement device of claim 1 further comprising:
tubing that is adjacent to the film wherein the tubing extends from the second aperture.

7. A method for assembling a securement device for use in negative pressure wound therapy wherein the securement device has a release strip, a release layer, a film with an adhesive and a back layer, wherein the release strip, the release layer, the film, the adhesive and the back layer are generally planar and generally parallel with respect to each other, the method comprising the steps of:
coating a top surface of the film with an adhesive wherein the film has a first aperture that extends through the film;
attaching the back layer to the film wherein the back layer covers the film;
securing the release layer to the adhesive wherein the release layer has a second aperture that extends through the release and that is larger than the first aperture and further wherein the release layer covers the adhesive such that a ring of the adhesive is exposed between the first aperture and the second aperture; and
securing the release strip to the ring of the adhesive wherein the release strip has a section that is unsecured to the ring of adhesive and that contacts the release layer.

8. The method of claim 7 further comprising the step of:
grasping the section of the release strip.

9. The method of claim 7 further comprising the step of:
pulling the section of the release strip in a direction away from the film to remove the release strip.

10. The method of claim 7 further comprising the step of:
attaching a suction port to the ring wherein the suction port has a flange that covers the ring.

11. The method of claim 7 further comprising the step of:
removing the release layer from the film.

12. The method of claim 7 further comprising the step of:
peeling the back layer from the film.

13. A securement device for use in negative pressure wound therapy, the securement device comprising:
a film having a top surface with an adhesive and bottom surface positioned opposite to the top surface wherein the film has a first perimeter and has a first aperture having a first diameter wherein the first aperture extends through the film;
a release layer secured to the film wherein the release layer has a second perimeter and is substantially parallel to the film wherein the release layer has a second aperture that extends through the release layer wherein the first perimeter and the second perimeter are aligned with respect to each other wherein the second aperture has a second diameter that is greater than the first diameter so that the film has a ring of adhesive that is exposed between the first aperture and the second aperture;
a release strip having a third aperture that extends through the release strip and having a third perimeter defined by a first edge, a second edge, a third edge and a fourth edge wherein the first edge is positioned opposite to the second edge and the third edge is positioned opposite to the fourth edge and further wherein the first edge and the second edge are generally perpendicular to the third edge and the fourth edge wherein the first edge and the second edge extend along the second perimeter wherein the release strip contacts the release layer and is adhered to the ring of adhesive and further wherein the release strip has a section that extends beyond the ring of adhesive and is unsecured to the ring of adhesive; and
a back layer having a fourth aperture that extends through the back layer wherein the back layer is secured to the film wherein the film is between the back layer and the release layer and further wherein the back layer has a fourth perimeter that is aligned with respect to the second perimeter.

14. The securement device of claim 13 further comprising:
tabs secured to the adhesive on the top surface of the film wherein the tabs are positioned on opposite ends of the film and further wherein the tabs are between the release layer and the back layer.

15. The securement device of claim 13 further comprising:
a suction port having a dome and a flange that extends from the dome wherein the flange is substantially parallel to the film and is adhered to the ring.

16. The securement device of claim 13 wherein the film is constructed from polyurethane.

17. The securement device of claim 13 wherein the ring is generally circular.

18. The securement device of claim 13 wherein the back layer is kiss cut so that the back layer is divided between a first side and a second side.

19. The securement device of claim 13 further comprising:
tubing that is generally cylindrical and that is adjacent to the film.

20. The securement device of claim 13 wherein the first perimeter and the second perimeter are substantially shaped as an oval.
